(19) United States
(12) Patent Application Publication
(10) Pub. No.: US 2002/0193885 A1
(43) Pub. Date: Dec. 19, 2002

(54) Prostheses for plastic reconstruction with improved hydrophilicity properties, and method for obtaining them

(75) Inventors: Gilbert Legeay, Saint Saturnin (FR); Christelle Porcheron, Chenu (FR)

Correspondence Address: YOUNG & THOMPSON, 745 SOUTH 23RD STREET 2ND FLOOR, ARLINGTON, VA, 22202

(73) Assignee: ASSOC. POUR LES TRANSFERTS DE TECHNOLOGIES DU MANS, LE MANS (FR)

(21) Appl. No.: 10/103,702

(22) Filed: Mar. 25, 2002

(30) Foreign Application Priority Data
Mar. 23, 2001 (FR) 0103977

(51) Int. Cl. A61F 2/02

(52) U.S. Cl. 623/23.72; 623/8; 623/14.13

(57) ABSTRACT

The present invention relates to the field of prostheses for plastic reconstruction, especially to mammary and muscular prostheses.

More specifically, the present invention concerns a prosthesis for plastic reconstruction with improved hydrophilicity properties, comprising an envelope composed of a base polymer material, characterized in that the base material of the envelope is modified on its surface by creation of polar sites and coated with a layer of at least one hydrophilic polymer.

### SCOPE OF THE INVENTION

The present invention relates to the field of prostheses for plastic reconstruction, especially to mammary and muscular prostheses.

More specifically, the present invention concerns a membrane for a plastic reconstruction prosthesis with improved hydrophilicity properties, as well as a method for obtaining this membrane or these prostheses.

### PRIOR ART

Prostheses for plastic reconstruction are most often composed of a pocket of flexible material of the elastomer type, in particular based on silicone or polyurethane, where appropriate inflatable using a system of valves, or filled with physiological serum before or after implantation.

In the prior art, there have been attempts to improve various properties of the external surface of prostheses for plastic reconstruction, particularly for mammary prostheses.

It has been known for a long time that the presence of prostheses inserted into living tissue causes the formation of retractile fibrous nodules which lead to a loss of flexibility of the breast after several months, and which can lead in some cases to rupture of the prosthesis membrane.

To resolve this problem, several methods for hydrophilization of the external surface of a silicone polymer have been reported in the prior art.

The PCT application No. WO 99/18886 proposes implants comprising an external layer of bio-resorbable material such as polyglycolic acid, polylactone, polycaprolactone or a synthetic resorbable lactomer marketed under the name “Polysorb” by the company UNITED STATES SURGICAL CORPORATION.

The European patent application No. EP 057 033 proposes two types of solutions, the first solution consisting of creating polar sites on the external surface of the silicone membrane of a mammary prosthesis, the second solution consisting of applying hydrophilic compounds onto this external surface.

The creation of polar sites on the surface of the prosthesis membrane, particularly by means of a plasma, would increase the hydrophilic nature of the surface of the envelope.

The European patent application No. EP 057 033 also discloses various methods for applying hydrophilic compounds to the envelope surface.

According to a first embodiment, hydrophilic monomeric compounds are chemically grafted by covalent bonds onto the envelope surface, on which reactive functions, for example Si-H groups, have previously been generated, the grafting being performed in the presence of a platinum catalyst.

According to a second embodiment, the reactive functions may be created on the surface of the silicone envelope by gamma radiation or low-wavelength ultraviolet radiation in order to activate the Si-CH₃ bonds, which allows the grafting of the hydrophilic monomeric compounds.

According to a third embodiment, a monomeric compound is deposited on the silicone envelope surface by vaporization in a vacuum chamber, followed by an in situ polymerization of the monomer, either via a plasma, or by appropriate irradiation such as ultraviolet.

The single example of patent No. EP 057 033 describes the hydrophilization of the surface of the silicone envelope by creation of polar sites using a source supplying oxygen-containing positive ions in a vacuum chamber.

The methods of hydrophilization of the prosthesis envelope surface, in particular mammary prostheses, described in the prior art have distinct disadvantages according to the type of method used.

Thus, it is shown according to the invention that the creation of polar sites on the surface of a prosthesis envelope is temporary and does not lead to a lasting improvement in the hydrophilicity properties imparted by these polar sites.

The chemical grafting of the hydrophilic monomeric compounds to the envelope surface requires the exposure of the surface of the prosthesis envelope to high-energy radiation, such as gamma or low-wavelength ultraviolet radiation, in order to generate the reactive functions involved in the creation of the covalent bonds between the envelope surface and the hydrophilic monomeric compounds. However, it is known in the state of the art that ion beams or energetic radiation penetrate deeply into the polymer material treated and generate chemically reactive functions within the body of the polymer material, and not only at the surface. This results in a measurable alteration of the structure of the polymer thus treated, leading in particular to additional crosslinking of the polymer, causing substantial modification to its mechanical properties by increasing its rigidity. Such modifications of the physical properties of the base polymer material of the envelope of the mammary prosthesis significantly reduce its plasticity, which is an essential property of the prosthesis.

In addition, the use of hydrophilic monomeric compounds introduces a further technical disadvantage for obtaining a final product with the qualities required for use in humans (pharmaceutical grade), because of the presence of ungrafted non-polymerized monomers or oligomers, which must thus imperatively be removed before the product can be used for plastic surgery.

Following the use of such prior art methods, it is thus necessary to show, for each batch of the final product, in other words after each chemical grafting or each in situ polymerization, that the products are not toxic. These checks are long and costly, and incompatible with industrial and standardized production of prostheses for plastic reconstruction.

The applicant has thus endeavoured to develop prostheses for plastic reconstruction with improved hydrophilicity properties and without the disadvantages of the prostheses described in the prior art.

### DESCRIPTION OF THE INVENTION

The object of the invention is a prosthesis for plastic reconstruction with improved hydrophilicity properties, comprising an envelope composed of a polymer material, characterized in that the base material of the envelope is modified on its surface by the creation of polar sites and coated with a layer of at least one hydrophilic polymer.

It has been shown according to the invention that a prosthesis for plastic reconstruction such as that defined above, in addition to its ability to be easily introduced into the mammary compartment as a result of its improved slipperiness properties, also facilitates the degassing of the mammary compartment after insertion, because of the better circulation of the air at the surface of the external envelope.

In addition, the hydrophilic nature of the prosthesis envelope significantly reduces the formation of fibrous nodules over time, probably because of the lower adhesion of the fibroblasts to the envelope surface.

The hydrophilic nature of the surface of the envelope of a prosthesis according to the invention also imparts mobility properties within the mammary compartment, after implantation.

The base polymer material of a prosthesis according to the invention is exclusively modified on the surface, without detectable modification to the structure of the body of the polymer nor to its mechanical properties. It has been shown according to the invention that the polar sites are localized on the surface of the polymer material, over a thickness of 5 to 20 nm, and on average about 10 nm.

The mammary prosthesis according to the invention thus retains the mechanical properties of plasticity of the base polymer material before its surface modification.

Overall the properties listed above of a mammary prosthesis according to the invention impart excellent plasticity and flexibility, properties which are particularly sought in plastic reconstruction surgery.

A prosthesis according to the invention is also characterized in that the layer of at least one hydrophilic polymer is maintained on a long-term basis on the envelope surface, because of the creation of the polar sites which increase the surface energy of the base polymer material constituting this envelope, and thus encourage the adhesion of the layer of at least one hydrophilic polymer via numerous weak bonds, such as hydrogen bonds, ionic attractions or by Van der Waals forces.

The layer of at least one hydrophilic polymer adheres, without chemical grafting, to the surface of the envelope material by the formation of non-covalent bonds between the polar sites created on the surface of the base polymer material and the hydrophilic groups of the polymer.

The base polymer material of the prosthesis is preferably a silicone polymer or a polyurethane, well known to a person skilled in the art.

The preferred silicone polymers are the polyalkylsiloxanes, and even more preferably polydimethylsiloxane.

The polyurethanes usable as base polymer materials of a prosthesis according to the invention are for example those disclosed in the patents U.S. Pat. Nos. 5,133,742, 5,229,431, 5,254,662 or 4,873,308.

However, the base polymer material of a prosthesis according to the invention is strongly preferred to be a silicone polymer.

The creation of the polar sites on the surface of the base polymer material of the envelope of a prosthesis according to the invention mainly corresponds to increasing the proportion of carbonyl, hydroxy or amine groups, and free radicals. The free radicals recombine with each other, or with the oxygen in the air, thus creating the polar sites.

The polar sites present on the surface of the base polymer material preferably comprise the following sites:

C=O, CH₃O, C₂H₃O, C₃H₇O, OH, C₂OH, C₈H₅O, NH, NH₂, NH₄⁺, C₂H₈N⁺

A prosthesis for plastic reconstruction according to the invention is also characterized in that the layer of at least one hydrophilic polymer which adheres without covalent bonds to the surface of the base polymer material of the envelope has a thickness of between 1 and 100 micrometers, preferably between 2 and 50 micrometers, and even more preferably of about 30 micrometers.

The thickness of the layer of at least one hydrophilic polymer is advantageously sufficient to impart a uniform hydrophilic character of the whole surface of the envelope of the prosthesis which remains stable over time, because the prosthesis for plastic reconstruction must remain in the body over the long term.

A layer of at least one hydrophilic polymer with a thickness of between 10 and 50 micrometers is particularly preferred, and even more preferably between 25 and 40 micrometers. Without wishing to be bound by any particular theory, the applicant considers that a thickness of the layer of at least one hydrophilic polymer greater than 50 micrometers, or even greater than 40 micrometers, although not presenting any particular technical disadvantage, is not justified to achieve the objectives sought by the invention.

According to a first embodiment of a prosthesis for plastic reconstruction according to the invention, the layer of at least one hydrophilic polymer coats only one of its two surfaces, preferably the external surface of the envelope which is in direct contact with the tissues at the site of the implantation.

It has been shown according to the invention that coating the internal surface of the envelope of the prosthesis, previously modified on the surface by the creation of polar sites, by a layer of at least one hydrophilic polymer allows a simpler, more rapid and more complete degassing at the time that the prosthesis is filled with a gel or a physiological saline solution, because of the improved circulation of air bubbles created at the time of filling. These properties are particularly advantageous when the prosthesis is filled in situ after implantation in the body.

Thus, in a second embodiment of a prosthesis for plastic reconstruction according to the invention, the internal surface of the polymer material of the envelope, previously modified on the surface by the creation of polar sites, is coated with a layer of at least one hydrophilic polymer.

According to a third embodiment, the external surface and the internal surface of the base polymer material of the envelope of the prosthesis are both coated with a layer of at least one hydrophilic polymer after creation of polar sites.

The hydrophilic polymer is preferably soluble in water. In fact, because the bio-artificial organ is implanted into a host organism, the use of organic solvents is excluded since their complete removal is difficult, and their presence, even in low quantities, is not compatible with therapeutic or surgical use in humans or animals.

The hydrophilic polymer material is preferably selected from the following hydrophilic polymers:

the celluloses and their derivatives, such as hydroxypropylmethylcellulose (HPMC), for example the HPMC E4M marketed by the Company DOW CHEMICALS, or that named Aquilon marketed by the Hercules Company, or the carboxymethylcellulose marketed by the Company DOW CHEMICALS;

the polyacrylamides and their copolymers, such as those marketed by the Company SIGMA (Uppsala, Sweden);

polyvinylpyrrolidone (PVP) and its copolymers, such as those marketed by the Company BASF/Laserson, such as Kollidon;

the copolymers of vinyl acetate, such as the copolymer of vinyl polyacetate and polyvinyl alcohol marketed under the name Mowiol by the Company HOECHST/CLARIANT;

the polyethylene glycols, such as those marketed by the Company SIGMA;

the propylene glycols;

the hydrophilic poly(meth)acrylates, such as those marketed by the Companies DEGALAN or DEGUSSA;

the polyosides;

the chitosans, such as those marketed by the Company SIGMA.

By hydrophilic polymer according to the invention should be understood either a polymer material composed of one of the hydrophilic polymers as defined above or a mixture of several of the hydrophilic polymers above, in general a mixture of two or three of the hydrophilic polymers above.

According to a first aspect, the prosthesis for plastic reconstruction as defined above consists of a mammary prosthesis or implant.

According to a second aspect, the prosthesis consists of an implant for muscular reconstruction.

According to a third aspect, the prosthesis consists of an implant for reconstruction of the testicles.

A further object of the invention consists of a method for obtaining a prosthesis for plastic reconstruction with improved hydrophilicity properties, characterized in that it comprises the following steps:

a) creation of polar sites on the surface of the base polymer constituting the envelope of the prosthesis;

b) dipping the envelope thus treated into an aqueous solution of at least one hydrophilic polymer; and

c) drying.

The creation of the polar sites on the surface of the envelope of the prosthesis is preferably performed by plasma treatment, by corona effect discharge, or by electromagnetic discharge at atmospheric pressure or under vacuum.

An oxygen, argon, nitrogen or carbon dioxide plasma is advantageously used.

The surface of the envelope is preferably treated with an argon radiofrequency plasma. It may be treated at a plasma reactor emission power of between 3 and 10 watts per liter of reactor capacity, for between about 1 and 20 minutes. The treatment also be performed by a microwave plasma, at the same power, but for 5 seconds to 20 minutes.

The plasma treatment is preferably performed in a vacuum or partial vacuum.

The pressure is preferably between 0.1 and 100 Pa, and more preferably between 1 and 50 Pa.

For performing a method of plasma treatment, the skilled person may advantageously refer to the book by André Ricard entitled “Plasmas réactifs” published by Editions SVF in 1995.

The treatment may also be performed by corona discharge. The voltage of the treatment is advantageously between 50 and 500 volts, the intensity being variable according to the treatment device and the items treated. The length of treatment is of the order of tenths of seconds, preferably between 0.1 and 1 second. In the case of continuous treatment, the length of exposure is such that the material to be treated passes across the treatment device at a speed of a few centimeters to several decimeters per second.

In addition, the prosthesis envelope may be treated several times to increase the effectiveness of the treatment.

The treatment by corona discharge may be performed using devices with opposite parallel electrodes, with side-by-side parallel electrodes (electrode arc about 5 mm high), or blown arc (side-by-side parallel electrodes with gas current between them, thus creating an electric arc about 10 cm high).

For performing a method of corona discharge or electromagnetic discharge treatment, the skilled person may advantageously refer to the book by André Ricard (1995) cited above.

The strongly preferred method for creating the polar sites is by a step of treatment with an argon plasma performed at a power of 50 watts for ten minutes.

Whatever the type of surface treatment applied out of those described above, the applicant has determined, by ESCA or XPS analysis and by abrasion by argon descaling, that the base polymer of the prosthesis is modified by creation of polar sites over a thickness of 5 to 20 nanometers, and in general over a thickness of about 10 nanometers.

The plasma treatment leads to an oxidation stable over time, particularly by creation of oxidant groups such as alcohol, acid and carbonyl groups which increase the hydrophilicity of the polymer material surface and thus also its surface energy. For example, the wetting index corresponding to the value of the angle (theta) taken at the point of contact of a drop of liquid with the surface of the silicone polymer constituting the envelope of a prosthesis onto which it is placed passes from about 100° before plasma treatment to about 50° after plasma treatment.

It has been shown according to the invention that the coating of the surface of the silicone envelope of a prosthesis, after creation of polar sites, by a layer of at least one hydrophilic polymer considerably increases its hydrophilic properties, since its wetting index observed after coating with PVP is less than 20°, in comparison with a value of about 100° for the wetting index observed for the envelope before treatment.

The step b) of coating the surface of the envelope of the prosthesis after creation of polar sites by a layer of at least one hydrophilic polymer may be performed by dipping, by application with a brush or by spraying from a gun.

The application of the hydrophilic polymer by dipping, advantageously for 1 second to 1 minute, preferably 5 seconds to 30 seconds, before draining and drying, is simple and rapid. This method is particularly well suited to large-scale production of prostheses according to the invention.

The preferred length of the dipping step is between 5 seconds and 10 minutes.

The dipping step advantageously takes place in an aqueous solution of the hydrophilic polymer at a temperature of between 15° C. and 25° C., preferably at laboratory temperature.

The drying step c) may be performed by any means known in the state of the art, preferably in air or in a ventilated oven.

The application of the hydrophilic polymer with a brush may be advantageous to coat small well defined areas of the prosthesis. The application with a brush, although it may be used to apply the polymer to the whole surface of the prosthesis to be treated, is preferably used to complement dipping, for example in cases where, in rare cases, the dipping step has not resulted in complete coating of the surface of the envelope to be treated.

The application of the hydrophilic polymer by spraying at atmospheric pressure leads to a dry film of the hydrophilic polymer more rapidly than by dipping or by application with a brush.

In a preferred embodiment of the method, the coating of the internal surface of the envelope of the prosthesis is performed before its assembly.

The methods for treating the internal envelope of the prostheses are preferably the plasma methods, since these can be used for objects with complex geometry and hollow objects.

Whatever the type of hydrophilic polymer used, the quantity of this polymer, in total weight of the solution, is preferably adjusted to obtain an aqueous solution of the hydrophilic polymer with a viscosity of between 1 and 10 centipoises, as measured according to the DIN rotary viscometer technique for liquids (equivalent to the Brookfield viscosity).

For example, a viscosity value of the order of 5 to 10 centipoises (cPs) is obtained by a concentration of 1% by weight of PVP (Kollidon K90 marketed by BASF) or by a concentration of 0.2% by weight of HPMC (E4M marketed by DOW CHEMICALS). The viscosity measurements were performed using a needle of the DIN 30D type, at ambient temperature and for a rotation speed of 300 to 500 r.p.m.

As an illustration, when the hydrophilic polymer is hydroxypropylmethyl cellulose (HPMC), polyvinylpyrrolidone (PVP) or a mixture of these two polymers, the percentage by weight of the hydrophilic polymer, with respect to the total weight of the aqueous solution of the polymer, is advantageously between 0.1% and 1%. The length of the step of dipping the polycarbonate film in a solution of hydrophilic polymer is adjusted so as to obtain a polymer layer with a thickness of between 10 and 100 nanometers.

The aqueous solution of at least one hydrophilic polymer contains either only or predominantly water as solvent, where appropriate in combination with one or more solvents completely miscible with water, preferably ethanol.

The aqueous solution of at least one hydrophilic polymer advantageously contains one or more antibacterial agents, preferably chosen from the silver salts, or the iodine or quaternary ammonium salts.

The aqueous solution of at least one hydrophilic polymer is preferably prepared and stored under sterile conditions.

All the steps of the method for obtaining a semi-permeable membrane according to the invention are preferably performed under aseptic conditions using sterile and if possible apyrogenic materials.

The method according to the invention advantageously contains an additional step of sterilization of the prosthesis, which may be performed either cold or hot by techniques well known to a skilled person.

As an illustration, the sterilization step may be performed using an autoclave, for example at a temperature of 121° C. for 20 minutes without causing significant alteration to the advantageous properties of the prosthesis.

The prosthesis may be stored under sterile conditions before use, for example in a physiological saline solution such as a 9% solution by weight of sodium chloride.

According to another aspect, the prosthesis of the invention may be stored dry, preferably at a temperature of about 4° C.

The invention is further illustrated, without in any way being limited, by the following figures and examples.

### EXAMPLES

### EXAMPLE 1

Production of a hydrophilic mammary prosthesis according to the invention.

For the production of a mammary prosthesis with improved hydrophilicity properties according to the invention, we used a prosthesis envelope made from silicone elastomer marketed by the Company PEROUSE PLASTIE under the reference TX or AX.

The prosthesis envelope was treated with an argon plasma in a chamber with a volume of 20 liters at a power of 50 watts for 10 minutes at a pressure of about 1 m/ and at a temperature close to ambient temperature. The discharge was of the capacitive type, at a frequency of 13.56 MHz.

After creation of polar sites on the surface of the silicone polymer material constituting the envelope of the prosthesis by the plasma treatment, the envelope was dipped for about 30 seconds in an aqueous solution of polyvinylpyrrolidone (PVP) marketed under the name Kollidon 30 by BASF/LASERSON.

After dipping, the envelope was drained and dried in an oven or in a flow of air, optionally heated.

### EXAMPLE 2

Study of the wetting indexes of the hydrophilic prostheses according to the invention.

1. Wetting measurement

The wetting index is given by the value of the angle (theta) taken at the point of contact of a drop of liquid with the surface of the polymer material on which it is placed. The figure corresponding to this angle is determined on one side by the straight line corresponding to the surface of the object, and on the other side by the tangent to the drop at the point of contact drop/surface of material.

For a hydrophilic surface, the drop is flat: the angle is small;

For a hydrophobic surface: the drop resembles a ball the angle is large.

Origin of the water:

The water used for the measurements was:

either deionized by ion-exchange resins;

or freshly distilled;

or commercially available: injectable preparation (ppi), pure water.

This water must be stored in closed quartz containers in the absence of light and heat.

The volume of the drop was a few microliters. Under these conditions, the weight of the drop was small, so that the drop did not significantly deform under the effect of gravity. This drop could be produced either from a micropipette (Pasteur type) or from a microsyringe.

### Apparatus Used

The size of the drop did not allow direct measurement of the angle of contact. It was necessary to use an optical apparatus. The possibilities were:

a photographic apparatus with macro lens;

a system of projection onto a screen with a system of graduation of angles;

a goniometer with enlarging telescope: on the market is equipment such as that marketed by the Company Ramé-Hart in the United States, by Kruss in Germany or by Kyowa in Japan;

a goniometer with camera and computer marketed by Kruss or by GBX Instruments in France (Digidrop).

It is desirable that the equipment is located in a room with constant temperature, close to 20° C.

The measurements given in the examples below were made with the Digidrop apparatus, according to the technique described below.

The drop was formed at the end of the syringe (or micropipette), then the test material was slowly approached to the drop (static drop). The image of the drop and the surface immediately appeared on the screen: the measurement of the angle was performed on this image.

The measurement could either be made automatically, or manually by pointing with the computer mouse at the two points of contact drop/surface and the top of the drop: the computer automatically calculated the value of the contact angle. It was preferable to perform the measurement manually, as reflection problems could sometimes interfere with reading the image by the camera when this was operated in automatic mode.

The measurement was performed about 5 seconds after the drop was deposited. For some hydrophilic surfaces, a gradual spreading of the drop occurred over time. In addition, the water tended to evaporate, which was however not perceptible in the 5 seconds after deposit.

For each surface, a minimum of three measurements was performed and the mean value was calculated together with the standard deviation.

The images and values of angles were recorded by the computer.

2. Results obtained with two hydrophilic polymers

The wetting indexes, represented by the values of the angle theta taken at the point of contact between a drop of liquid and the surface of the material tested, were measured, comparing the silicone polymer before and after plasma treatment followed by coating with the hydrophilic polymer.

As initial material, we used a silicone envelope of a prosthesis marketed by PEROUSE PLASTIE under the reference TX or AX.

The plasma treatment and the coating with a layer of polymer were performed as described in example 1.

### Results Before Treatment

Before any treatment, the value of the angle was measured as from 95° to 105° for the silicone material, showing the hydrophobicity of the surface of this film.

### Results After Plasma Treatment

After treatment with argon plasma, the value of the angle was between 38° and 51° depending to the test. The creation of polar sites on the surface of the silicone had thus strongly increased the surface energy and the hydrophilic character of the silicone material constituting the envelope of the prosthesis.

Results after plasma treatment, then application of a hydrophilic polymer layer.

1) After coating of the silicone polymer, pre-treated with argon plasma, with a layer of PVP (1% aqueous solution), the value of the angle passed from 44° to 51° (after plasma treatment) to 16° to 18°.

2) After coating of the silicone polymer, pre-treated with argon plasma, with a layer of hydroxypropylmethylcellulose (1% aqueous solution), the value of the angle passed from 38° to 45° (after plasma treatment) to 60° to 78°.

The layer of hydrophilic polymer thus significantly increased the hydrophilic properties of the envelope of the hydrophilic prosthesis.

It has thus been shown that the surface of a prosthesis according to the invention had hydrophilicity properties significantly increased in comparison with those of the untreated prosthesis.

In addition, the retention of the hydrophilic polymer on the internal and/or external surface of the prosthesis, due to the creation of polar sites, enabled the hydrophilicity properties to be retained over a long period.

### EXAMPLE 3

Comparison of the variation over time of the hydrophilicity properties of a silicone surface treated with plasma, with or without a laver of hydrophilic polymer.

For this comparative study, the envelope of a mammary prosthesis in silicone polymer was treated according to the protocol described for example 1.

A first silicone membrane was subjected to argon plasma treatment only.

A second silicone membrane was first treated with argon plasma before deposit of a layer of polyvinylpyrrolidone (PVP).

The wetting index of the surface of each membrane treated as described above was measured according to the protocol described for example 2.

The wetting index measurements were performed just after treatment, then from 4 hours to 7 days after the treatment, respectively after storage in dry conditions at ambient temperature in the presence of silica gel (less than 2% RH) or in a damp atmosphere at ambient temperature in the presence of a hydrated salt (65% RH).

The results are given in table I.

The results in table I show that the plasma treatment, because of the creation of polar sites, significantly increased the hydrophilicity properties of the silicone envelope. However, these hydrophilicity properties imparted by the plasma treatment were completely lost 4 hours after the treatment, whether the silicone envelope was stored in a dry or a damp atmosphere.

In contrast, a prosthesis membrane according to the invention, treated with plasma then coated with a layer of PVP, retained its hydrophilicity properties over time.

The retention of the hydrophilicity properties of a prosthesis membrane according to the invention could especially be observed when the membrane was stored in a damp atmosphere, since only a small increase of the wetting index could be observed seven days after the treatment, it being noted that, by definition, a prosthesis for plastic reconstruction is intended to be maintained in a damp atmosphere during its use in the body of the patient.

TABLE I
Comparison of the wetting indexes
of two prosthesis envelopes as a function
of time after treatment

| | Wetting index at different times after | | | |
|---|---|---|---|---|
| | treatment | | | |
| Product tested | 0 | 4 hours | 24 hours | 7 days |
| Untreated silicone envelope | 87° | — | — | — |
| Silicone envelope treated with | 27° | — | — | — |
| argon plasma | | | | |
| Dry storage | — | 95° | 104° | 104° |
| Storage in damp atmosphere | — | 80° | 102° | 102° |
| Silicone envelope treated with | 22° | — | — | — |
| plasma + PVP | | | | |
| Storage in dry atmosphere | — | 23° | 24° | 45° |
| Storage in damp atmosphere | — | 21° | 21° | 31° |

1. Prosthesis for plastic reconstruction with improved hydrophilicity properties, comprising an envelope composed of a base polymer material, characterized in that the base material of the envelope is modified on its surface by creation of polar sites and coated with a layer of at least one hydrophilic polymer having a thickness of between 1 and 100 micrometers, preferably between 2 and 50 micrometers, and even more preferably of about 30 micrometers.

2. Prosthesis according to claim 1, characterized in that the hydrophilic polymer or polymers are selected from the celluloses and their derivatives, the polyacrylamides and their copolymers, polyvinylpyrrolidone (PVP) and its copolymers, the copolymers of vinyl acetate and vinyl alcohol, the polyethylene glycols, the propylene glycols the hydrophilic poly(meth)acrylates, the polyosides and the chitosans.

3. Prosthesis according to one of claims 1 and 2, characterized in that the base material is modified on only one of its two surfaces.

4. Prosthesis according to one of claims 1 to 2, characterized in that the base material is modified on both surfaces, internal and external.

5. Prosthesis according to one of claims 1 to 4, characterized in that the base polymer material of the envelope is a polyorganosiloxane, in particular a silicone elastomer, or a polyurethane.

6. Prosthesis according to one of claims 1 to 5, characterized in that it is a mammary implant.

7. Prosthesis according to one of claims 1 to 5, characterized in that it is an implant for muscular reconstruction.

8. Prosthesis according to one of claims 1 to 5, characterized in that it is an implant for reconstruction of the testicles.

9. Method for obtaining a prosthesis according to one of claims 1 to 8, characterized in that it comprises the following steps:
a) creation of polar sites on the surface of the base polymer material constituting the envelope;
b) coating the surface of the base material thus treated with a layer of at least one hydrophilic polymer; and
c) drying.

10. Method according to claim 9, characterized in that the step a) is performed by a plasma treatment, by corona effect discharge, or by electromagnetic discharge at atmospheric pressure or under vacuum.

11. Method according to one of claims 9 or 10, characterized in that, in step b), the hydrophilic polymer or polymers are selected from the celluloses and their derivatives, the polyacrylamides and their copolymers, polyvinylpyrrolidone (PVP) and its copolymers, the copolymers of vinyl acetate and vinyl alcohol, the polyethylene glycols, the propylene glycols the hydrophilic poly(meth)acrylates, the polyosides and the chitosans.

12. Method according to one of claims 9 to 11, characterized in that step b) is performed with an aqueous solution of at least one hydrophilic polymer which has a viscosity of between 1 and 10 centipoises.
